(19) United States
(12) Patent Application Publication
(10) Pub. No.: US 2022/0074586 A1
(43) Pub. Date: Mar. 10, 2022

(54) Thin Film Capillary Vaporization: Device and Methods

(71) Applicant: Vapor, LLC, Concord, CA (US)

(72) Inventors: Lars Barfod, Danville, CA (US); Robert L. Connick, Walnut Creek, CA (US); William F. Connick, Walnut Ceek, CA (US)

(21) Appl. No.: 17/280,310

(22) PCT Filed: Sep. 25, 2019
(86) PCT No.: PCT/US19/53019

§ 371 Date: Mar. 26, 2021

Related U.S. Application Data
(60) Provisional application No. 62/736,469, filed on Sep. 26, 2018.

(51) Int. Cl. F22B 1/28; A24F 40/10; A24F 40/44; A24F 40/46; A24F 40/51; A24F 40/48; H05B 1/02; A61L 9/03

(52) U.S. Cl. CPC F22B 1/288; A24F 40/10; A24F 40/44; A24F 40/46; F24F 6/18; A24F 40/48; H05B 1/0244; A61L 9/037; A24F 40/51

(57) ABSTRACT

The present invention relates to an apparatus and method for the generation of directed vapor from a liquid source. Vaporization takes place within a device capable of confining boiling to a geometrically small volume, and expelling it as heated vapor via capillary vaporization. The foregoing is accomplished through the use of a lightweight compact and portable personal vaporization device that generates heated vapor by the flash boiling of small volumes of aqueous liquid in a safe and energy-efficient manner. The flash boiling takes place at the interface between a disk heater and a non-fibrous wick that receives liquid at one surface and generates vapor that is collected and pressurized in grooves at an opposing surface. In an alternate configuration, a heat distributor may be used between the heater and wick. The apparatus and methods are directed toward personal humidification for comfort and therapeutic purposes in the case of aqueous liquids, but may also be used with other, non-aqueous liquids.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

### 1. Field of the Disclosure

The present disclosure describes improvements to modules and methods for the low energy flash-like vaporization of liquids. More particularly, the disclosure relates to improved thin film capillary force vaporizer modules for the flash boiling of small volumes of aqueous liquid to heated vapor in a safe and energy-efficient manner. Such capillary force vaporizer modules are particularly well suited for use in lightweight, compact and portable personal vaporization devices.

### 2. Discussion of the Related Art

One class of modules that can be used to generate vapor from a liquid are known as capillary pumps, capillary vaporization modules or capillary force vaporizers (CFVs). These units generate pressurized vapor directly from unpressurized liquid by applying heat to cause liquid to boil within a capillary member, and by at least partially constraining the evolved vapor to allow pressure to increase. The pressurized vapor then exits the CFV through one or more orifices at a high velocity. Such modules are thermally powered, compact, and generally have no moving parts, thereby offering several advantages over other techniques used for liquid vaporization and vapor pressurization. Capillary force vaporizer modules and devices in which they may be found are variously described in commonly-owned U.S. Pat. No. 5,692,095 to Young, issued 25 Nov. 1997, U.S. Pat. No. 5,870,525 to Young, issued 9 Feb. 1999, U.S. Pat. No. 6,162,046 to Young, et al., issued 19 Dec. 2000, U.S. Pat. No. 6,347,936 to Young, et al., issued 19 Feb. 2002, U.S. Pat. No. 6,585,509 to Young, et al., issued 1 Jul. 2003, U.S. Pat. No. 6,634,864 to Young, et al., issued 21 Oct. 2003, U.S. Pat. No. 7,431,570 to Young, et al., issued 7 Oct. 2008, U.S. Pat. No. 7,920,777 to Rabin, et al., issued 5 Apr. 2011, U.S. Pat. No. 7,942,644 to Young, et al., issued 17 May 2011, U.S. Pat. No. 8,201,752 to Brodbeck, et al., issued 19 Jun. 2012, and U.S. Pat. No. 9,746,194 to Brodbeck, et al., issued 29 Aug. 2017. The foregoing are incorporated by reference herein.

For decades, physicians for a wide range of medical conditions have recommended the vaporization of liquids for humidification purposes. Physicians regard humidification as part of “supportive care,” that is, an intervention that helps to relieve flu-like symptoms and provide comfort to a patient in addition to rest, fever control, and hydration. Consumers have found humidification also useful and beneficial for various aspects of personal care. For instance, humidification can aid in skin moisturization, cleansing and in personal hydration. A number of the personal humidification devices described above have enjoyed increasing acceptance by consumers over various prior art humidifiers for these and additional purposes. At the heart of each humidifier referenced above is a capillary force module, alternately referred to herein as a capillary force vaporizer or CFV.

As often is the case with consumer products, the longer an item is placed into service, the more is learned about the reliability and functioning of that product. Often, situations arise that may not have been observed or even anticipated during the development of that item. Such is the case with the capillary vaporizer modules, or CFVs, described herein. For instance, as the manufacture, assembly and materials used in CFVs have improved, it has been observed that several factors may impact the useful lifetime of the CFV module. One issue relates to failing of the wick used to deliver liquid from a reservoir to the receiving surface of a CFV to be vaporized. Over time, it has been observed that poor wicking of liquid feed can put a great deal of strain on the CFV heater to the point of rendering the vaporizer module of a personal humidifier inoperable, or perhaps worse. Another issue relates to the durability of the heater. An additional factor concerns the means for providing power to the heater while simultaneously providing mechanical means to retain and align CFV component parts.

### SUMMARY OF THE INVENTION

The instant disclosure describes efforts to overcome certain limitations of, and make improvements to, the prior art by providing improved capillary force vaporizers, or CFVs, for the vaporization of liquids and the pressurization of vapor. A number of aspects of prior CFV design, materials and manufacturing techniques were studied in greater depth with an eye towards improvements and greater reliability of CFV modules overall. For instance, in the past, delivery or transport wicks were often fabricated from fibrous materials, such as nylon resin, which by nature is lipophilic, i.e., repels water. Such wicks work well when used with oily or fatty substances, such as those which might be used in oil lamps or fragrancing devices, but they do not readily facilitate the transportation of aqueous feeds. In order to improve the delivery of aqueous feeds, prior transport wicks were often treated with a hydrophilic coating either during production of the starting resin, after the wicks were fabricated or, in certain cases, at both points during production of the wicks. Use of hydrophilic coatings necessitated additional steps to heat treat or bake the hydrophilic coatings onto the wicks.

Unfortunately, with the passage of time, it was found that problems occasionally arose with each of the foregoing wick coating approaches. For one thing, the process used to coat wicks was found to change from batch to batch as well as among wicks within a given batch. The process for coating the wicks, in addition to increasing manufacturing costs, introduced additional variability in the final CFV modules. Controlling the coating process proved to be difficult and prone to inconsistencies. Finally, with greater longevity of CFV-based humidifiers, it was occasionally found that the coating was not durable. The coating was observed to wear off with time and repeated use of the humidifier. In fact, portions of the coating were detected on the surface of the CFV as deposited material.

With loss of the transport wick's hydrophilic coating, delivery of water to the CFV can become increasingly erratic, and may eventually become insufficient. Regardless of the level of feed water in the reservoir and the age of the humidifier, water delivery to the CFV module should remain constant and consistent at all times. If it is not, heater temperatures can exceed 100° C. or become erratic, thereby putting stress on the heater's physical stability. Deposition of any extraneous material onto surfaces of the CFV during operation of a humidifier within which it is housed should be minimal. Accordingly, there was a desire to improve the materials and configuration used to deliver a liquid to a CFV module for vaporization in a personal humidifier.

A second area for improvement in CFVs concerned the heaters used to vaporize the liquid feed. Among the CFV references cited above, CFV heaters were typically fabricated from ceramic discs onto which a resistive paste was applied in such a manner as to create a somewhat serpentine resistive trace. Several layers of a glass coating material in turn covered the heat trace to insulate it from possible corrosion by the water. On the reverse side of the heater disk, an intricate furrowed or grooved pattern was created to channel the steam that was generated towards a centrally located orifice in the heater.

Over time, it was found that the glass coating on the upper surface of the heater trace would occasionally develop cracks. Once formed, the cracks in the glass coating would ultimately result in corrosive destruction and failure of the heat resistance layer. The cause for the cracks was attributed to temperature swings experienced in heating and rapid cooling of the glass coating.

The grooved or channeled underside surface of the heaters also proved to be problematic over time. Thickness differences of the ceramic as between channels and vanes disposed there between occasionally resulted in uneven heating and cooling of the thin ceramic, i.e., thermal stress, which could ultimately result in breakage or cracking of the heater. Accordingly, improvements in the heater configuration and manufacture were desired.

Problems were also occasionally experienced when spring clips were used as a mechanical force generator or means to hold the heater and porous member together in CFV modules. Spring clips were employed to simultaneously hold the heater and porous member in heat exchanging contact, as well as provide electrical power to the resistive heater. The clips were often fabricated from gold-plated stainless steel, in order to provide long durability, corrosion resistance, and to facilitate connecting power wires directly to the clips. Unfortunately, as CFVs experienced increased use and ran for greater lengths of time, corrosion of the clips started to appear with greater frequency. Without being bound by theory, it was hypothesized that with the passage of direct current (DC) power through the electrical leads to the clips over time, even minimal mineral content in the distilled water feed could cause electrolysis to take place and therefore corrode the clips. In addition, the application of electricity to open connectors could place additional requirements on mechanical construction to avoid electric shock to a user. Accordingly, there was a desire to pursue modifications in the technique(s) and materials used for delivery of electrical power to CFVs.

It is therefore desirable to provide a device that can provide humidification without requiring the heating of large quantities of water while permitting the generation of water vapor in a short time. In particular, it is desirable to deliver therapeutic or beneficial heated humidity to an individual in a manner that is safe, efficient and can be accomplished quickly. In particular, it is desirable to provide devices for personal humidification that employ capillary force vaporizer modules or CFVs that incorporate improvements in longevity of service without some of the accompanying configuration and materials problems of prior art CFV devices. Accordingly, the present disclosure describes apparatuses and method for the safe, nearly instantaneous generation of pressurized water vapor from non-pressurized liquid feeds that exhibit improvements in reliability and durability over capillary force vaporizers of the prior art.

### BRIEF DESCRIPTION OF THE DRAWINGS

FIG. 1 is a schematic plan view showing the combination of a heater, porous member and wick of a prior art capillary force vaporizer module. Both upper and lower surfaces of the heater are illustrated.

FIG. 2 is a cross sectional view of a CFV module according to one aspect of the present invention.

FIG. 3 is an oblique view of a different wick according to another aspect of the present invention.

FIG. 4 is an oblique view of a still different non-fibrous wick according to a different aspect of the present invention.

FIG. 5 is an illustration of one side of a commercially available ring heater.

FIG. 6 is an oblique view of a partial CFV module according to yet another aspect of the present invention.

FIG. 7 is an illustration of one side of a smooth non-fibrous heater according to yet another aspect of the present invention.

FIG. 8 is an oblique illustration of a smooth non-fibrous heater and a long wick according to yet a different aspect of the present invention.

FIG. 9 is a schematic plan view of a portion of a capillary force vaporizer module according to yet another aspect of the present invention, showing the combination of a smooth heater, grooved non-fibrous wick and temperature sensor.

FIG. 10 is a graph of temperature (° C.) versus time (sec) showing heating profiles for two CFV assemblies in a first Rapid Cooling Test.

FIG. 11 is a graph of temperature (° C.) versus time (sec) showing heating profiles for two different CFV assemblies in a second Rapid Cooling Test.

FIG. 12 is a graph of temperature (° C.) versus time (sec) showing heating profiles for yet two different CFV assemblies; one with an embedded temperature sensor as compared to one with a resistive heater.

FIG. 13 is an oblique illustration of a CFV module according to an alternate aspect of the present invention.

### REFERENCE NUMERALS USED IN THE FIGURES

100 Prior art CFV module
103 Heater thickness
104 Upper surface
105 Diameter
106 Orifice
108 Heat trace
109 Terminus
110 Heater
112 Electrical leads
120 Porous member
130 Wick
140 Heater underside
150 Lower surface
152 Grooves/channels
200 CFV module
202 Mechanical force generator
204 Electrical lead
206 Solder
210 Holder
212 Ledge
214 Foil
216 Ground
300 Long non-fibrous wick
302 Upper portion
304 Lower portion
310 Wick
400 Grooved long non-fibrous wick
402 Upper surface
404 Raised portion
406 Channels
408 Groove
410 Wick
412 Upper portion
414 Lower portion
500 Heater
510 Ring heater
552 Opening
554 Heater diameter
556 Opening diameter
558 Electrical lead
600 Partial CFV module
610 Heat distributor
612 Orifice
630 Plate clamp
700 Heater
702 Top surface
704 Bottom surface
706 Leads
708 Orifice
710 Disk heater
800 CFV portion
810 Partial CFV module
900 CFV portion
910 Temperature sensor lead
912 Temperature sensor
1300 CFV module
1302 Tab
1304 Mechanical force generator
1306 Lower portion
1308 Engagement means
1309 Arms
1310 Span
1312 Attachment site

### Definitions

In the present specification and claims, reference will be made to phrases and terms of art which are expressly defined for use herein as follows:

Vaporizer as used herein is understood to refer to a device for converting liquid substances into vapor form. While the thin film vaporizers discussed and described herein are primarily used for converting water or medicated liquid into vapor for inhalation, moisturization, or for providing and supplying or maintaining humidity, the vaporizers described and contemplated for use herein may also be used with other liquids. Such liquids may include alcohols, oils, fragrance oils and other non-aqueous liquids as well as combinations of any of the foregoing.

### DETAILED DESCRIPTION OF THE INVENTION

The capillary force vaporizers that are described herein feature certain improvements over similar capillary pumps and capillary vaporizers of the prior art. The improvements that will be described herein concern modifications to the main components of a CFV, namely: the wick, the heater, and the mechanical force generator that has been used in past CFV modules to hold components of a CFV together. In order to appreciate the improvements that have been made to the capillary vaporizers described herein, some background information may be helpful.

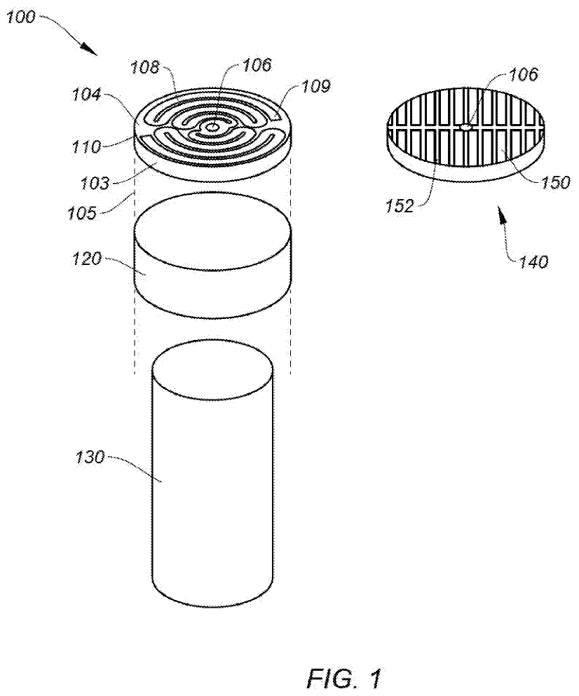

In the past, capillary vaporizers were regarded as comprising components similar to those presented schematically in FIG. 1. FIG. 1 illustrates a sample CFV device of the prior art at 100. It should be noted that like numbers are used throughout the description to represent common elements. Accordingly, device 100 includes heater 110, porous member 120 and wick 130. Heater 110, in turn, includes heat trace 108 provided on a first side or upper surface 104 of heater 110 and also includes at least one orifice 106 for the release of pressurized vapor that is generated by the CFV. Heater 110 is a disk having a side 103, the height of which is much smaller than heater diameter 105. Heat trace 108 is comprised of a resistive paste, which in turn is covered by layers of glass (not shown). Heat trace 108 also includes at least one terminus 109. Terminus 109 provides a site that may be used for the attachment of one or more electrical leads, for situating one or more mechanical force generators to provide compression among the components of a CFV or for any combination of the foregoing. Heater 110 also includes underside or lower surface 150 shown at 140 in FIG. 1. In addition to orifice 106, lower surface 150 of heater 110 also includes a plurality of grooves, furrows or channels 152. Channels 152 are used to collect vapor that is generated during operation of the CFV, and permits pressurization of the vapor until it is released through orifice 106. Electrical leads 112 (not shown) are used to connect heat trace 108 to a source of electrical power for the CVF.

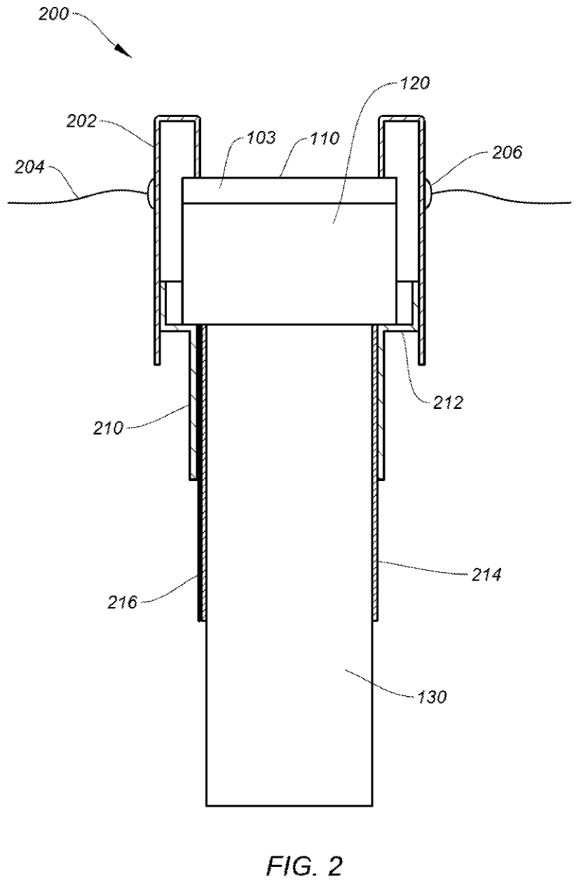

Adjacent to and situated in heat-exchanging contact with heater 110 is porous member 120. Porous member 120 also has a diameter 105 that is essentially the same as that of heater 110. Liquid feed that is to be vaporized by the CFV is supplied to porous member 120 via wick 130. Wick 130 has a diameter that is slightly less than diameter 105 of heater 100 and porous member 120 in order to facilitate placement of wick 130 into a holder or collar (not shown in FIG. 1) or in order to provide a ledge or anchoring site for a clip or other means for placing heater 110, porous member 120 and wick 130 together in a stacked configuration. A sample holder 210 is shown in cross section at 200 in FIG. 2.

During operation of CFV modules, heat is provided to porous member 120 by heater 110. A liquid to be vaporized is drawn by capillary forces from a reservoir (not shown) to a region of porous member 120 at or near the interface between porous member 120 and heater 110 where it is vaporized. Vapor generated from porous member 120 is collected builds pressure within vapor collections grooves or channels 152 situated on underside 150 of heater 110, thereby forcing the generated vapor out through orifice 106. Past observations have indicated that the geometric configuration of liquid that is heated to vaporization may be described as a thin liquid film or simply a thin film. Consequently, CVFs may also be referred to as thin film vaporizers. Despite their small and compact size, thin film vaporizers of the type described herein are typically capable of vaporizing at least 2.0 g/min water.

### Improved Wick

As mentioned above, a number of issues came to light with ever increasing lifetimes of personal humidifiers in which CFVs are incorporated. One area concerned the liquid delivery component or wicks. With time, poor wicking of the liquid feed due to breakdown of the hydrophilic material used to coat wick fibers and/or finished wicks caused decreasing delivery of aqueous feed to CFVs. Reduced liquid feed, in turn, negatively impacted reliability and performance of the CVFs. Ideally, whenever a CFV is in operation, the delivery of aqueous feed should remain constant, regardless of the water level in the water reservoir of the humidifier or the age of the device.

Wicks and other liquid feed components that have been used with CFVs in the past have variously been described in commonly owned U.S. Pat. No. 5,870,525 for liquid fuels as well as U.S. Pat. Nos. 6,585,509, 6,634,864 and 7,431,520 for aqueous liquid feeds. Materials that have been used for wicking material included: polymeric aramids such as KEVLAR™ felt and braid as well as cotton-fiberglass such as NOMEX™, all available from E.I. DuPont de Nemours & Co., Delaware. Nylon wicks from Porex of Fairburn, Ga., were also used. Porous ceramics such as alumina grindstone was from Abrasives Unlimited Inc., San Leandro, Calif.; other porous materials for use as porous members are available from Refractron of Buffalo, N.Y.; and from Xiamen Innovacera Advanced Materials Co., Ltd of Xiamen, Fujian, China, as brown or white porous alumina and silicon nitride ceramics. In the course of the instant work, a number of studies were undertaken in order to improve upon the use and configuration of the foregoing materials and methods for delivery of feed liquid to CFVs.

In one series of studies, over-size wicks having larger bundles of fibers were fabricated in order to enable the flow of more liquid feed than was needed. In this manner, as the wick became less hydrophilic over time, there would still be a sufficient quantity of aqueous feed available for use by the CFV. While this approach met with some success on a laboratory scale, problems still persisted. Thicker wicks proved to be harder to coat with hydrophilic coatings and variability in the coating thickness of the hydrophilic layer proved to be unavoidable. Moreover, the coatings were still found to break down with time, and coating residues continued to appear on heater surfaces.

In another series of studies in an effort to retard the degradation and failure of prior art wicks over time, CFV modules were constructed as shown schematically in cross-section at 200 in FIG. 2. Thus, the CFV module illustrated at 200 includes heater 110, porous member 120 and wick 130, similar to a CFV of the prior art shown at 100 in FIG. 1. In addition to a heater, porous member and wick, CVF 200 also shows the approximate location of a spring clip or mechanical force generator at 202, electrical leads 204 and holder 210. Electrical leads 204 are joined in electrical contact with mechanical force generator 202 via some means of connection, which in FIG. 2 is provided by solder 206. In addition to providing a contact site for electrical power, mechanical force generator 202 also provides a means for containing heater 110 and porous member 120 in heat-exchanging contact with each other. Mechanical force generator 202 further provides compressive force between heater 110 and holder 210 through the use of side arms disposed towards the bottom of force generator 202 (not shown) that engage tabs on the side of holder 210 (also not shown).

Unlike CFV 100, however, CFV module 200 also includes foil 214 and ground 216. As will be understood by those knowledgeable in the relevant field of art, ground 216 may have various shapes and forms such as, but not limited to, the following: a pin, wire, plate, foil, or combination of any of the foregoing, etc. In some aspects, ground 216 is a grounding pin. Materials that are suitable for use as ground 216 include aluminum, steel and copper. Ground 216 may be either coated or uncoated. In a preferred aspect, ground 216 is a gold-plated stainless steel pin.

When in use in combination with a CFV, foil 214 wraps around wick 130 and is disposed between holder 210 and wick 130. The purpose for foil 214 and ground 216 is to protect a total dissolved solids (TDS) apparatus that is situated in the liquid reservoir of a vaporizer in which the CFV is used. Foil 214 and ground 216 help to shield the TDS apparatus from high voltage and current in the heater. As will be readily appreciated by those knowledgeable in the relevant art, measuring the electrical conductivity of water is a common technique for measuring total dissolved solids in the water. Foil 214 and ground 216 therefore also indirectly protect heater 110, spring clips 202 and wick 130 by shutting off power to the CFV if excessive minerals are detected. This could occur, for example, in cases where saline solution or mineral-rich water is used as a liquid feed in a CFV instead of distilled water. The use of foil 214 and ground 216 can also help to reduce harmful stresses on heater 110 by detecting the absence of water electrically, rather than monitoring for temperature fluctuations or temperature excursions at heater 110. It was also anticipated that use of foil 214 and ground 216 would help cut down on some of the degradation of fibrous wick 130 that has been observed to occur over time and continued use of CFVs. However, even when distilled water was used exclusively as the liquid feed for CFVs under controlled circumstances, a small but finite amount of pitting or corrosion of spring clips takes place. Despite all the foregoing modifications, neither wick 130 nor CFVs of the type shown at 200 were able to resolve some of the underlying issues that seemed to plague CFVs. It just was not possible to consistently and reliably operate a stable CFV over extended time periods.

Ultimately, it was decided to try a different and unique approach to the traditional methodology for CFV liquid delivery. Realizing that the porous member performed rather flawlessly in conducting liquid feed from the wick to the heater, it was decided to completely eliminate the fibrous wick that had traditionally been used to draw liquid from a CFV reservoir to the porous member. Instead, it was decided to completely eliminate the fibrous wick and replace it with a unitary non-fibrous, ceramic member. At the time, it was not known whether or a unitary member could effectively be employed to draw liquid from a liquid reservoir and deliver it to a CFV heater situated at some distance from the reservoir for vaporization. In order to test this approach, a series of new non-fibrous wicks or “long ceramic wicks” were fabricated and evaluated.

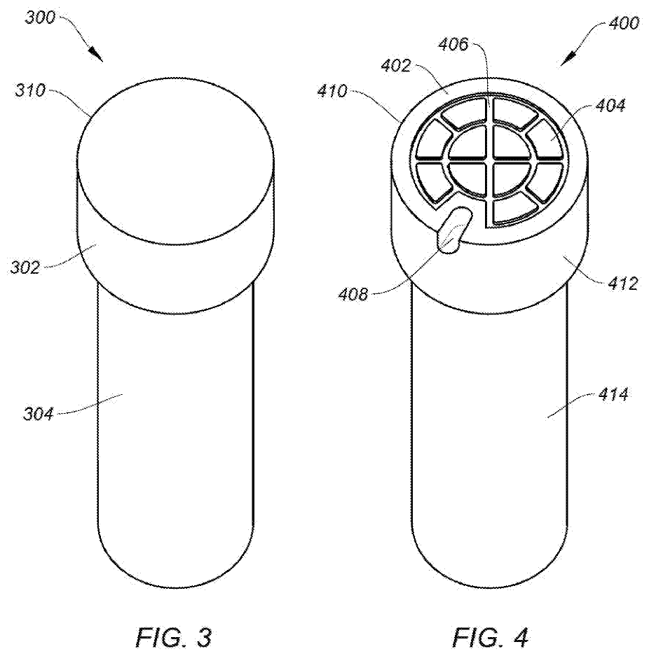

One such long wick, also referred to herein as a non-fibrous wick, is shown in FIG. 3 at 300. Accordingly, long wick 310 includes upper portion 302 and lower portion 304, which are shown as having slightly different diameters. In practice, this difference in diameters is not necessary. Both upper portion 302 and lower portion 304 may have the same diameter. For purposes of the instant disclosure, having different diameters for upper and lower portions of the ceramic wick simply permitted continued use of holders 210. Materials that are suitable for use with the long, non-fibrous wicks described herein include: porous ceramics such as alumina grindstone from Abrasives Unlimited Inc., San Leandro, Calif.; brown alumina ceramics manufactured by Refractron Technologies Corporation of Newark, N.Y.; other porous materials for use as long wicks are available from Xiamen Innovacera Advanced Materials Co., Ltd of Xiamen, Fujian, China, as brown or white porous alumina and silicon nitride ceramics.

Criteria for selecting a material suitable for use in non-fibrous wick 310, in addition to the requirement that the material feature a range of pore sizes, include timed water absorption. Water absorption was evaluated by comparing the dry weight of short ceramic wicks of the prior art with the weight of that wick after contact with water supplied to the short ceramic wick via a fibrous wick, also of the prior art, for 5 seconds. Water absorption of long non-fibrous wicks was evaluated by submerging 10 mm of the long wick into distilled water and measuring the time from submersion to complete saturation of the wick. Over the course of numerous trials, it was found that a good wick gets completely saturated within about 20 seconds. Attention was also paid to the consistency of water absorption among possible long ceramic wick candidates. Thus, anywhere from 20 to 100 samples were evaluated for each of the most promising long or non-fibrous ceramic wicks.

Upper portion 302 of wick 310 is somewhat similar in height and diameter to porous member 120 of prior art CFV 110. In likewise fashion, lower portion 304 of wick 310 is somewhat similar in height and diameter to fibrous wick 130 of CFV 110. The overall size and volume of extended wick 310 is therefore similar to the combined dimensions and volumes of porous member 120 and wick 130 of prior art CFV devices, although variations in these measurements are possible. Accordingly, the total height of long non-fibrous wick 310 is generally less than about 5.0 cm. Upper portion 302 may have a radius of about 1.0 cm and a height of 1.0 cm, while lower portion 304 may have a height less than about 3.5 cm and a slightly smaller radius of less than 0.8 cm. According to another aspect, wick 310 has a lower portion 304 that is 33 mm long and 16 mm in diameter. In yet another aspect, long ceramic wick 310 has a lower portion 304 that is 52 mm long and 12 mm in diameter. Typically, the total volume of extended ceramic wick 310 is therefore less than 15.0 cm³.

The fact that upper portion 302 and lower portion 304 of wick 310 are shown as having different diameters at 300 in FIG. 3 is not crucial for use or inclusion of wick 310. As will be readily understood by those knowledgeable in the relevant field, upper portion 302 and lower portion 304 of long non-fibrous wick 310 can also have the same diameter, in which case wick 310 will appear as a cylinder of unitary diameter, and the total volume of wick 310 will be somewhat greater than 15.0 cm³. The fact that long non-fibrous wick 310 is shown as having two different diameters herein is purely a matter of convenience. With two sections having different diameters, wick 310 fits within holder 210 of prior art CFVs once porous member 120 and wick 130 are removed and substituted by long wick 310 in their stead. Accordingly, upper portion 302 of wick 310 can engage and rest on ledge 212 of holder 210, while lower portion 304 of wick 310 will project away from the heater to reach the liquid reservoir in the region previously occupied by wick 130.

Surprisingly, the approach of using a unitary wick for drawing liquid feed from a reservoir and delivering it to a CFV for vaporization in the course of the work described herein was found to be advantageous. First, from a manufacturing standpoint, it was found that no coatings were required to either modify or improve the hydrophilicity of ceramic wicks that were evaluated herein. Secondly, long non-fibrous wicks were unexpectedly found to perform at least as well as, if not better than, the combination of well-functioning fibrous wicks used in combination with short so-called porous members of the prior art. This was found to be the case even where extended ceramic wicks 310 were called upon to vertically transport aqueous feed distances on the order of several inches from a liquid feed reservoir to a CFV heater for vaporization. It was also unexpectedly found that the capillary feed characteristics in long wicks 310 evaluated herein experienced no significant change in performance either initially or over time. This greatly simplified the process for providing components for use with CFV modules for personal humidifiers, as well as the actual assembly of CFV devices.

Briefly, therefore, a capillary force vaporizer according to one aspect of the instant disclosure is a portable device or method for generating pressurized vapor from unpressurized liquid, comprising:
1. a non-fibrous wick comprising a capillary network, a surface for receiving liquid and a vaporization area in which vapor is produced from the liquid; and
2. a heater to convey heat to the non-fibrous wick, the heater component also including a series of grooves or channels for the collection and pressurization of vapor and at least one orifice for the release of the pressurized vapor.

In practice, a means for situating a non-fibrous or long ceramic wick in heat-exchanging contact with a heat source for vaporizing liquid feed is also appropriate. Accordingly, a capillary force vaporizer according to a different aspect of the instant disclosure is a device or method for generating pressurized vapor from unpressurized liquid, comprising:
1. a non-fibrous wick comprising a capillary network, a surface for receiving liquid and a vaporization area in which vapor is produced from the liquid;
2. a heater for conveying heat to the non-fibrous wick, the heater also including a series of grooves or channels for the collection and pressurization of vapor and an orifice for the release of the pressurized vapor; and
3. means for situating the non-fibrous wick in heat-exchanging contact with the heater.

As will be discussed in greater detail below, means for positioning a long or elongated non-fibrous wick in heat-exchanging contact with a heater component in a CFV can be achieved according to any of a number of techniques, examples of which have been described previously. See, for example, commonly-owned U.S. Pat. Nos. 7,920,777, 8,201,752, 9,746,194 and U.S. Ser. No. 12/095,481.

### Improved Heater

A second area of concern for increased longevity of capillary vaporizers that developed over time concerned the heating system. Heat is provided to a CFV to vaporize water or other aqueous feeds, thereby generating the steam or vapor that is provided by a hand-held vaporizer. Heaters that have been used most recently with CFVs comprise a thin ceramic disk with an orifice that is coated with a resistive paste and layers of glass on one side. One such typical heater is illustrated at 110 in FIG. 1 as discussed above. Again, upper surface 104 shown at 100 in FIG. 1 includes resistive paste, while lower surface 150 shown at 140 in FIG. 1, in addition to orifice 106, also includes a plurality of channels or groves 152. The intricate pattern of grooves 152 collects and channels steam that is generated at the interface between heater 110 and porous member 120. As the generated steam is collected in grooves 152 of heater 110, it builds pressure until it is released from the CFV at orifice 106.

As indicated above, several problems were found to occur with CFV heaters over time. Even though the heater need only provide sufficient energy to heat water to boiling (100° C., 212° F.), rapid heating or cooling can stress the thin glass coating on heat trace 108. Stress cracks can develop due to temperature fluctuations at the heater. Over time, even very small, hairline cracks in the coating can result in destruction of the resistance layer and therefore ultimately lead to the failure of heater 110. In addition to problems with the heat trace and glass coating, the substrate ceramic disk can also fail due to thermal stresses. The presence of grooves 152 on heater underside 150 result in the presence of greatly different material thicknesses in an already relatively thin ceramic piece. The result is that thin and thick portions of heater 110 expand and contract at different rates with temperature changes in the heater. This would occasionally lead to complete failure of the heaters (see below).

A number of approaches were pursued in order to provide more robust heaters and greater heater longevity for CFVs. Efforts to improve heater durability have included approaches such as the application of additional layers of glass coating onto the heat trace, increasing the purity of the alumina ceramic from which the substrate heaters are manufactured, modifying baking temperature profiles of the ceramic during manufacture, evaluating different types of glass coating materials for high temperature stabilities, etc. Unfortunately, none of the foregoing approaches proved to be either sufficiently reliable or sufficiently effective for stemming the underlying problem of heater breakage.

In one approach, it was decided to invert the configuration of the heater disk and porous member, such that the channels that are used to conduct steam towards the heater orifice are implemented in the porous member, rather than at the underside of the thin heater. This change can be viewed schematically with reference to FIGS. 3 and 4. Recall the discussion above concerning the former combination of a porous member and separate fibrous wick. These individual components can be considered to have been merged into a unitary, elongated or non-fibrous wick as described above with respect to 300 in FIG. 3. Introduction of a series of channels to the top surface of wick 310 gives rise to a grooved long non-fibrous wick, which is illustrated at 400 in FIG. 4. Thus, grooved wick 410 includes upper portion 412 and lower portion 414. And just as the diameters of upper portion 302 and lower portion 304 of non-fibrous wick 310 can have the same or different diameters, the same holds true here. Upper portion 412 and lower portion 414 of grooved wick 410 can also have diameters that are similar, or which may be different. The only difference between wick 310 and wick 410 are the absence or presence of additional features on their upper portion, respectively. Accordingly, a complex pattern of channels 406 are provided at upper surface 402 of non-fibrous wick 410, resulting in a plurality of raised portions 404. Also shown cut into an edge of upper surface 402 of upper portion 412 of grooved wick 410 is optional groove 408.

By way of example, therefore, in still another aspect, a capillary force vaporizer as contemplated herein may be regarded as a device or method for generating pressurized vapor from unpressurized liquid, comprising:
1. a non-fibrous wick 310 further comprising a capillary network, a surface for receiving liquid and a vaporization area in which vapor is produced from the liquid;
2. a heater component for conveying heat to the non-fibrous wick, the heater component further including a plurality of channels for the collection and pressurization of vapor that is produced from the liquid and at least one orifice for release of the vapor at a velocity greater than zero; and
3. means for situating non-fibrous wick 310 in heat-exchanging contact with the heater component.

In another configuration, a capillary force vaporizer according to another aspect may be regarded as a device or method for generating pressurized vapor from unpressurized liquid, comprising:
1. a non-fibrous wick 410 further comprising a capillary network, a surface for receiving the liquid and a vaporization area in which vapor is produced from the liquid, and further including a plurality of channels for the collection and pressurization of vapor that is produced from the liquid;
2. a heater component for conveying heat to the non-fibrous wick, the heater component further including an orifice for release of the vapor at a velocity greater than zero; and
3. means for situating wick 410 in heat-exchanging contact with the heater component.

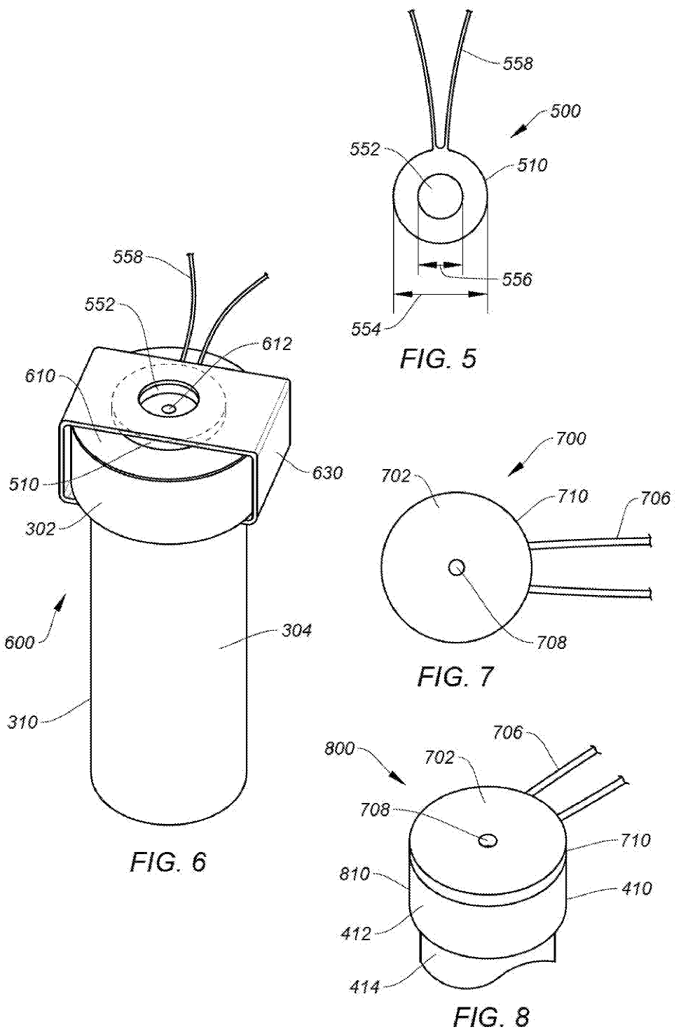

Having made the case to eliminate heater grooves from the underside of prior art CFV heaters, heaters that resulted were still ones that retained a glass-coated heat trace on a first flat surface of the ceramic disk substrate and a smooth surface on an opposing flat surface of the ceramic disc substrate. The question then arose as to whether or not it was necessary to maintain the silk-screened, glass over-coated heat trace on an exterior surface of the heater component. After all, except for the exterior heat trace, the heater component was now more analogous in appearance to commercially available small ring heaters on the market. One such so-called “smooth ring” heater is illustrated schematically at 500 in FIG. 5. Thus, ring heater 510 includes an embedded resistive heat element (not shown) connected to electrical leads 558 disposed towards a side of the thin ring heater. Ring heater 510 also includes orifice 552 having a diameter 556 that is approximately half an order of magnitude larger than the opening of orifice 106 of heater 110, and exhibits an outer diameter 554 that is approximately 25% smaller than diameter 105 of any of heater 110, porous member 120, upper portion 302 of long wick 310 or upper portion 412 of grooved long wick 410. Commercially available smooth ring heaters such as 510 in FIG. 5 are made of alumina ceramic with embedded iron resistive material and associated leads. Such heaters are available from several suppliers, one of which is Fujian Minhang Electronics, Ltd., from Fujian, China.

A series of CFVs were assembled that employed commercially available ring heaters of the type 510 shown in FIG. 5. One problem that was experienced when using a commercially available ring heater in direct contact with a long non-fibrous wick that has no grooves, however, was that there was no way to either direct the vapor that was generated during operation of the CFV module towards the orifice of the heater, or to permit pressure to build up in the device. Wider orifices such as 552 found in ring heater 510 are less able to contain and build pressure in the vapor prior to being emitted from the CFV. Another problem was that in the absence of any vapor channels in either of the wick or heater 510, steam was unavoidably emitted around the periphery of heater 510 and leaked out around the outer edges of the ring heater where it contacted long wick 310, rather than being directed towards orifice 552.

After a number of trials, an approach that was found to successfully ameliorate the above non-directed steam discharge situation was to use heat distributor 610, disposed between ring heater 510 and long ceramic wick 310. Following a series of experiments, a thin heat distributor, such as a steel plate, was determined to perform most efficiently and effectively in this capacity. As will be readily understood by those knowledgeable in the relevant art, nearly any heat conductive material can be used for heat distributor 610, provided that it efficiently transfers heat and is not subject to thermal cracking at the temperatures at which a CFV module will operate. This, in turn, depends on the liquid feed that is supplied to the CFV for vaporization purposes. In general, materials that are suitable for use in heat distributor 610 may be selected from among stainless steels, metals, heat-conducting ceramics, heat-conducting polymers, as well as combinations of any of the foregoing.

A sample partial CFV including ring heater 510, a steel plate as heat distributor 610 and long non-fibrous wick 310 is illustrated at 600 in FIG. 6. With reference to FIG. 6, heat distributor 610 also includes orifice 612. Orifice 612 is narrower in diameter than is orifice 552 of ring heater 510, which is thus helpful in channeling vapor generated by the CFV into a narrower plume than if the heat distributor was not present. The CFV shown at 600 also includes a mechanical force generator in the form of plate clamp 630, which provides compression among ring heater, heat distributor 610 and wick 310, and also engages wick 310 along a lower edge of upper portion 302 of long wick 310. Electrical leads 558 of ring heater 510 are also shown. Note that the use of ring heater 510 with an embedded heat trace and its own electrical leads differs from prior art CFV modules in that mechanical force generator plate clamp 630 need not also function as a means for electrical connection to the heat trace of ring heater 510. Also, the type of mechanical force generator used with a CFV module of the type shown at FIG. 6 need not be of the box clamp variety, nor need it require a holder such as 210 shown in FIG. 2 in order to provide compression among the wick, heater and heat distributor. That is, as long as some provision for attachment of a mechanical for generator is provided, a CFV module does not necessarily require the presence of holder 210. Thus, the difference in outer diameters of upper portion 302 and lower portion 304 of long wick 310 provide a ledge that can be used for box clamp 630 to provide compression among ring heater 510, heat distributor 610 and long wick 310.

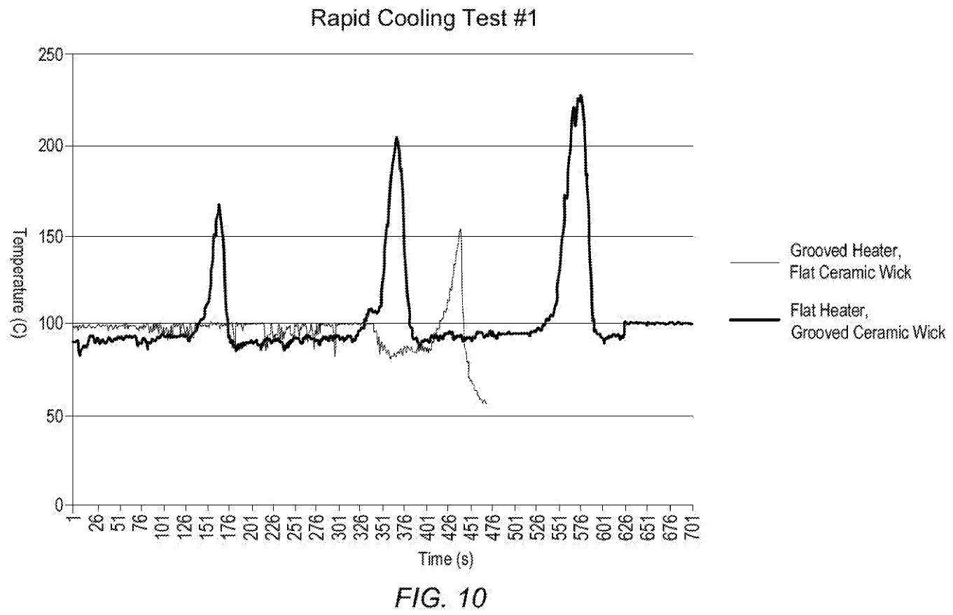

Considering heat distributor 610 further, the use of this component was advantageously found to provide a number of benefits in CFV assembly and operation. First, heat distributors 610 could act as a ground for heaters 510 as well as a heat sink to dissipate heat from the heater more evenly across the entire upper surface of long ceramic wicks 310. Without being bound by theory, it is believed that a heat distributor can slow down heat transfer from the heater to the non-fibrous wick. Heat distributor 610 thereby acts as a heat buffer, such that a heater does not experience extreme temperature gradients, as may occur when a CFV starts up and liquid feed is initially drawn towards the heater. In this context, recall the above discussion of the traces presented in the graph in FIG. 10. Even more importantly, the heat distributor helped to significantly reduce the amount of steam that was escaping from under heater 510, resulting in more steam being emitted via orifice 612 of heat distributor 610 and passing through orifice 552 of ring heater 510. In terms of improved heater longevity and efficiency, additional benefits were also realized with the ring heater plus heat distributor combination.

Without being bound by theory, one reason for the improvement of heater reliability and longevity may be due to the internally disposed heat trace of ring heater 510 as compared to surface heat trace 108 of heater 110. Even with a protective glass coating, heat trace 108 can experience wider temperature variations during CFV operation. Exposed heat trace 108 can be cooled via exposure to water condensation during operation of heater 110, while this is not possible with heater 510 due to its embedded resistive heat element. Consequently, there can be greater fluctuations in operating temperatures with the former, as compared to the latter. Using an embedded element heater can therefore reduce the frequency of observed thermal cracking of prior art heaters 110.

In a still different approach to solving the problem of premature heater failure, flat or plate heaters with embedded heat traces or embedded heaters were developed that had larger diameters than commercially-available ring heaters of the type shown at 510, while at the same time providing smaller orifices for the release of pressurized vapor. Element 700 shown in FIG. 7 illustrates one such heater. Thus, disk heater 710 includes an embedded heating element (not shown), electrical leads 706 and orifice 708. Top surface 702 and bottom surface 704 (not shown) are essentially smooth and featureless. Orifice 708 is again smaller in diameter than is orifice 556 of ring heater 510; the diameter of orifice 708 is on the order of one to two millimeters in size. Likewise, the outer diameter of heater 710 is greater than diameter 554 of ring heater 510. In fact, the diameter of 710 is preferably the same as the diameter of the porous member or the long wick with which it is used. One advantage to using disk heater 710 with its larger diameter as opposed to ring heater 510 is that the use of a heat distributor to more evenly distribute heat across the face of a porous member or non-fibrous wick can be completely eliminated (see discussion below).

A partial CFV that includes heater 710 in combination with a grooved long wick is illustrated schematically at 800 in FIG. 8. Thus, partial CFV module 810 depicts flat disk heater 710 with orifice 702 and electrical leads 706 situated atop wick 410, which in turn comprises upper portion 412 and lower portion 414. An advantage to using disk heater 710 together with non-fibrous wick 410 is two-fold. First, having a grooved pattern at the top surface of long wick 410 means that vapor can be collected, concentrated and directed towards orifice 708 of heater 710. Secondly, as the upper diameter of wick 410 and heater 710 are more closely matched, vapor does not escape out the sides of device 810 between the heater and the wick. This obviates the need for having to include a heat distributor such as 610 in FIG. 6.

In still another aspect, therefore, an improved capillary force vaporizer as contemplated herein comprises:
1. a grooved non-fibrous wick 410 comprising a capillary network, a surface for receiving liquid and a vaporization area in which vapor is produced from the liquid and further including a plurality of channels for the collection and pressurization of vapor that is produced from the liquid;
2. a heater 710 including an embedded heating element for conveying heat to the grooved wick, the heater further including at least one orifice for release of the vapor at a velocity greater than zero; and
3. means for situating the heater and grooved wick in heat-exchanging contact with one another and for providing compression among the heater and wick.

In yet a different aspect, an improved thin film capillary vaporizer as contemplated herein comprises:
1. a non-fibrous wick 310 comprising a capillary network, a surface for receiving liquid and a vaporization area in which vapor is produced from the liquid;
2. a heat distributor, the heat distributor further including means for the collection and pressurization of vapor that is produced from the liquid;
3. a heater 710 including an embedded heating element for conveying heat to the wick, the heater further including at least one orifice for release of the vapor at a velocity greater than zero; and
4. means for situating the heater, heat distributor and non-fibrous wick in heat-exchanging contact with one another. The means for the collection and pressurization of vapor that is produced from the liquid may be selected from among no orifice, one orifice or a plurality of orifices, further wherein the orifice may comprise a hole, groove, slit, slot, channel and combinations of any of the foregoing.

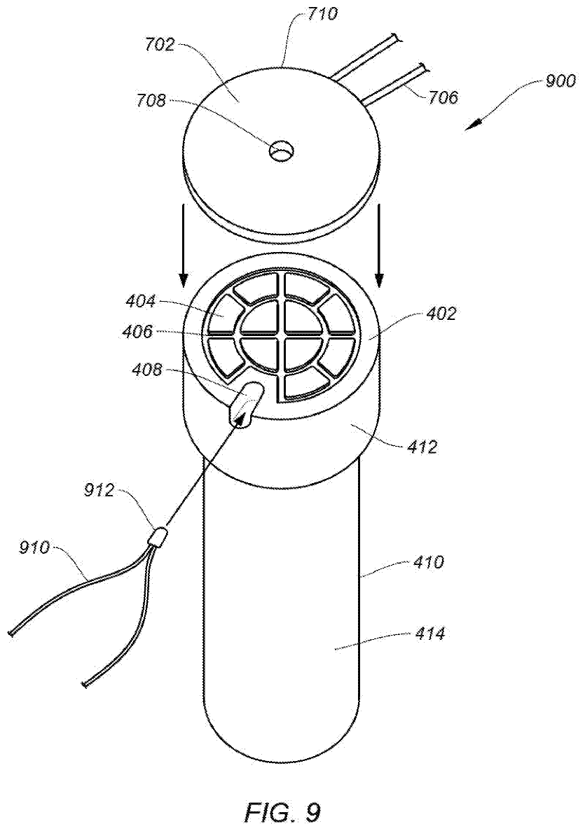

It was then realized that different CFV module configurations were possible that provided improved longevity and greater reliability in terms of performance over time than prior art CFV modules. One configuration described herein comprises the combination of a flat heater and a long non-fibrous wick, as shown schematically in FIGS. 8 and 9. Notice that in the different view of these combinations, shown at 800 and 900, respectively, the diameters of heater 710 and wick 310 are essentially the same. Also notice that the size of orifice 708 of heater 710 is comparatively small. Furthermore, the non-fibrous wick used in the foregoing combinations can be either of the smooth upper portion 302 type as shown for extended wick 300, or contain a grooved upper portion 412 as shown for extended wick 410 in FIG. 9.

As mentioned at several times in passing above, a mechanical force generator is often used with CFV devices in order to contain and provide compressive forces among the components of a CFV. At a minimum, these components typically, although not necessarily, include a heater and the combination of a porous member and fibrous wick or, as newly described herein, a heater and an elongated or long non-fibrous wick. One of the most common and convenient forms of a mechanical force generator that has been used with CFV modules over time is a spring clip. FIG. 2 includes one form of a spring clip at 202. The spring clip serves dual purposes of both holding CFV components together and serving as a point for electrical connection to the heat trace. Before the use of an embedded element heater, corrosion at the spring clip was an ongoing problem. Different types of steel, including steels that are manufactured especially for use in corrosive aqueous environments such as saltwater applications, were tried and evaluated for use as potential spring clips. Regardless of the type of steel used and the clip configuration, however, nothing successfully prevented electrolysis and corrosion from taking place at the spring clips. The clips corroded due to electrolysis, even where there was minimal mineral content in distilled water used in the CFV. Regardless of the material or technique tried, the spring clips continued to degrade, ultimately destroying whatever clips were tried. With time, they all simply failed.

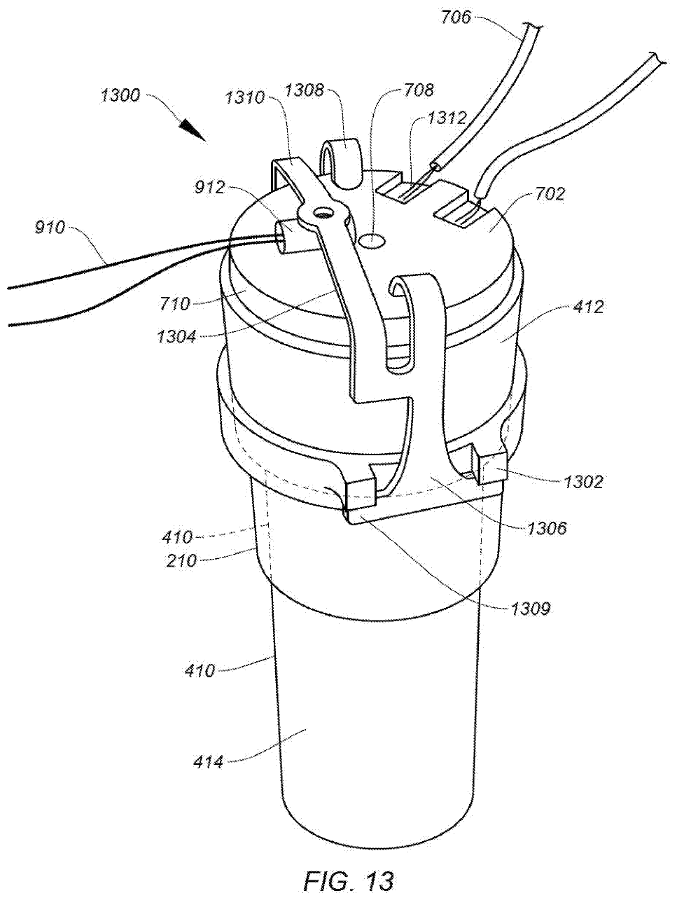

A distinct advantage of using a heating element with an embedded resistive heating element in a CFV module as described herein is that the connection of the power leads to the heater may also be enclosed. Embedded electrical connections are no longer exposed to the ambient atmosphere and are therefore less likely to corrode or fail with time. Alternately, placing the exposed voltage on a heater surface distant from wick 410 advantageously situates leads 706 in a dry environment, rather than at the base of the CFV, where water can collect. The role of the mechanical force generator used with CFVs is then simply to provide mechanical means for situating the heater in direct contact with the porous member or non-fibrous wick. Consequently, there are no constraints placed upon the shape or configuration of clips that can be used as fastening means in order to contain the elements of a CFV module together in heat-exchanging capacity. An embodiment of a CFV with an alternate form of mechanical force generator is shown in FIG. 13 at 1300 and described more fully after the discussion of Example 3, below.

Fortunately, once vapor collection and pressurization grooves were moved from the heater component to either prior art porous members or the elongated non-fibrous wicks described herein, it was found that CFVs began exhibiting longer reliable lifetimes. The relative numbers of heaters that failed due to cracking of the ceramic decreased noticeably. This observation was further supported by a series of stress-cracking studies. Accordingly, a number of CFVs were evaluated as described below.

### Example 1

One set of CFVs were assembled that combined grooved heaters, such as prior art heater 110, with smooth top surface, non-fibrous wicks 310 as newly presented herein. The foregoing may be regarded as having configuration A, that is, “grooved heaters-flat long wicks.” The performance of the foregoing assemblies were compared to a second set of CFV assemblies that included flat heaters, as represented by 710, in combination with grooved non-fibrous wicks 410 presented herein. These latter assemblies may be regarded as having configuration B, that is, “flat heaters-grooved long wicks.” The performance of the two CFV configurations are presented in the two traces shown in FIG. 10.

FIG. 10, which shows a graph of temperature (° C.) versus time (sec) illustrates a temperature profile obtained for the rapid cooling of two different CFV configurations, A and B, as described above. This series of experiments were designed to intentionally thermally stress the different heater configurations, in order to attempt to force the heaters to fail. The different traces of FIG. 10 illustrate what happens when a CFV, with no liquid initially present in the liquid reservoir, is operated at normal temperatures and at higher temperatures than would normally be used in the presence of an aqueous liquid feed. In a series of experiments, CFV modules were started, power was provided to the heaters causing them to heat up, and water at ambient temperatures was then added. Temperatures at the heaters were measured by placing a thermocouple on the top of the heaters. The addition of water caused the heaters to cool down rapidly; the temperature gradient of the heater material thereby induced thermal stress to the heaters.

In the series of experiments described above, CFV assemblies of type A, that is, grooved heaters-flat long wicks, also called grooved heater-flat ceramic wicks, gave rise to rapid cooling traces exemplified by the thin trace in FIG. 10. When room temperature water was added to Type A CFV assemblies that were heated to 150° C., the grooved heaters typically cracked apart and failed once water was made available to the CFV. By contrast, note the he heavy trace in FIG. 10. The heavy trace is characteristic of results that were obtained in rapid cooling tests using flat heater-grooved long wick or flat heater-grooved ceramic wick CFV assemblies of type B. Surprisingly, the type B CFV assemblies could be heated not only to 150° C., but to 200° C. and even as high as 225° C. and then be cooled rapidly with the introduction of water to the long wick, without failure of the heater. Note that even after several cycles involving elevated temperature excursions, CFV configurations of type B remained functional and continued operating at 100° C., the boiling point of water and therefore the typical operating temperature when aqueous feed is used with a CFV. To summarize these studies, note the difference in results obtained for the two CFV configurations presented in FIG. 10. Type A, grooved heater-flat wick CFV assemblies that included prior art heaters 110 failed repeatedly at 150° C. under rapid cooling test conditions. By contrast, Type B CFV assemblies of the grooved wick 410-flat heater 710 variety newly presented herein consistently withstood temperatures in excess of 150° C. under rapid cooling test conditions with no heater failure.

It was then realized that different configurations of CFV modules were possible that not only as demonstrated improved longevity and similar, if not more reliable CFV performance over time than prior art CFV modules. One configuration described herein comprises the combination of a flat heater and a long wick, as shown schematically in FIGS. 8 and 9. Notice that in these combinations, shown at 800 and 900, respectively, the diameters of heater 710 and wick 310 are essentially the same. Also notice that the size of orifice 708 of heater 710 is comparatively small. Furthermore, the non-fibrous wick used in the foregoing combinations can be either of the smooth upper portion 302 type as shown for long wick 300, or contain a grooved upper portion 412 as shown for long wick 410 in FIG. 9.

An alternate CFV configuration can also be contemplated as described herein. In cases where a CFV module comprises a heater and an elongated non-fibrous wick in which a heater has a smaller outer diameter than the diameter of the wick, a heat distributer can be advantageously disposed between the heater and the wick. In such instances, it has been found that many variations in configuration of the heat distributor are possible. In fact, a number of alternate configurations for openings or orifices in the heat distributor have been found to function quite satisfactorily. Thus, a heat distributor may contain one small central orifice, or may contain any number of additional openings. The openings may be arrayed around a central orifice or distributed around a central point of a heat distributor. An orifice need not even be centrally located. Alternate possible configurations include multiple grooves cut into one surface of the heat distributor or channels that completely penetrate the heat distributor. These geographical features may communicate with a central orifice, or simply consist of a series of grooves or channels with no particular orifice whatsoever. The grooves or channels can be either interconnected or isolated from one another. In general, if the wick that is used for a CFV contains a grooved upper surface, as shown at 410 in FIG. 9, it has generally been found preferable to use a heat distributor that features a single central opening, with no additional grooves or geography, in order for vapor generated at the upper surface of the wick to be most effectively and efficiently emitted from the CFV. In cases where a CFV contains a wick with a smooth upper surface and no channels, as shown at 310 in FIG. 3, then a heat distributor with a plurality of orifices and/or a plurality of channels that completely penetrate the heat distributor can also provide vapor and function acceptably within a CFV. All of the foregoing configurations, as well as others not mentioned here, have been tried and evaluated for use in CFVs and have found to provide acceptable functionality.

### Example 2

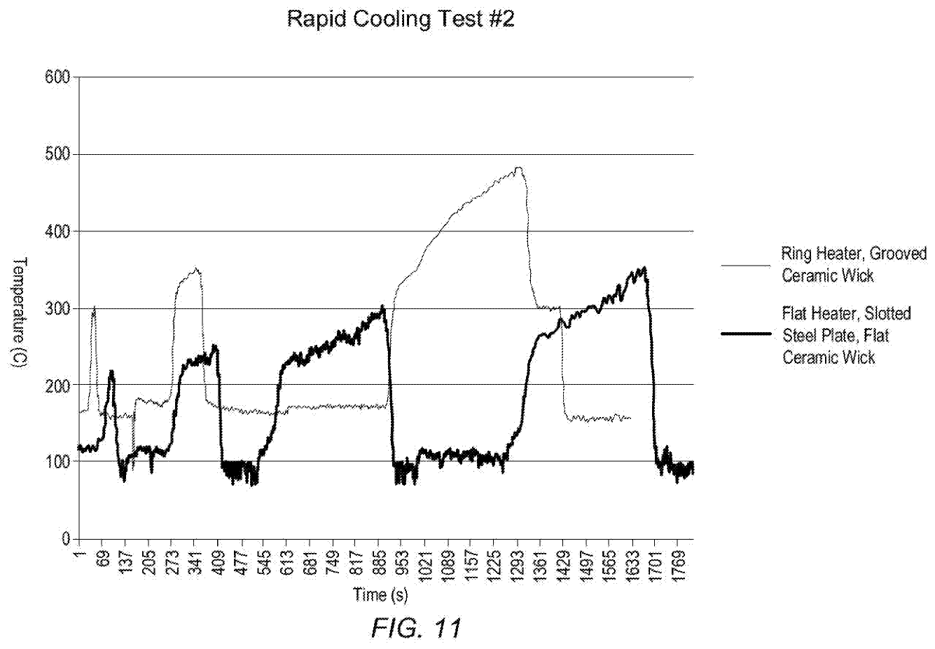

In order to further evaluate CFV performance with configuration variations, a different series of studies were undertaken. FIG. 11 shows the results obtained for CFV modules having heaters of different diameters, with heat distributors and grooved or smooth non-fibrous wicks, that were subjected to rapid cooling tests as described previously. Thus, the lighter trace in FIG. 11 shows the performance typical for modules of a Type C. Type C CVF modules comprised a ring heater such as that shown at 510 in FIG. 6, in combination with a heat distributor and a long, grooved non-fibrous wick. The heat distributor and wick had nearly the same outer diameters. while the outer diameter of the ring heater was smaller, similar to the configuration shown at 600 in FIG. 6. The heavier trace in FIG. 11 was obtained with CFV modules of a Type D. Type D modules comprised a flat or disk heater such as that shown at 710 in FIG. 9, a heat distributor and a smooth wick such as 310 shown in FIG. 3. The diameters of the heater, heat distributor and wick upper portion used in D-type assemblies were the same. Note that each of the heater and the upper portion of the wick used in Type D CFV modules were smooth. As such, there is no place for vapor to collect and become pressurized prior to being emitted through the orifice of the heater. Accordingly, the heat distributors that were used in Type D modules contained slots or channels cut through the heat distributors arranged in a series of parallel cords connected along a perpendicular, bisecting channel nearly as long as a diameter of the heater. These slots or channels provided spaces where vapor generated by the CFV module could be collected and pressurized, in the absence of having grooves on the upper portion of the non-fibrous wick. Different configurations, such as no holes, one hole or many holes or spaces in the heat distributor were evaluated, as well. Materials of construction that were evaluated for use with heat distributors contemplated for use herein have been copper and stainless steel. As will be understood by those knowledgeable in the relevant art, any material that can provide good thermal conduction may also be used.

In the examples shown in FIG. 11, the CFVs, which typically run at 40-45 Watt, were pushed to run to over 250° C. in the absence of water. Once water was added, the Type D CFV modules—which included a slotted heat distributor—cooled down to the normal 100° C. operating temperatures for water, without failure. Although the Type C CFV modules (light trace) contained ring heaters with smaller surface areas than the Type D CFVs, it was somewhat surprising to find that these modules ran at higher temperatures than the Type D CFVs (heavy trace). Without being bound by theory, it is suspected that the small ring heaters used in the Type C CVFs are simply worse at transferring heat to the heat distributor and eventually the non-fibrous wick due to their smaller surface area. More importantly, perhaps, these studies revealed how heaters having imbedded heating elements and smooth outer surfaces can reliably run at very high temperatures, without failure of the device or the heater, in particular. Even with a smaller diameter ring heater, as in the Type C devices, it was possible to run the CFV modules up to temperatures in excess of 450° C.(!) for over five minutes (1293 less 953 seconds), without a single heater failure in a CFV device. This remarkable result was totally unexpected.

Use of a heat distributor with CFV modules also provides a heat source for a thermal fuse, which may optionally be included in vaporizers that employ CFVs. Furthermore, separating the function of electrical connection of the mechanical force generator among the CFV components means that there is no need for exposed wires or electrical clips in the vicinity of liquid feed or water vapor, which lessened the likelihood of electrolysis taking place at clips 202. This, in turn, greatly reduced the amount of corrosion that had been experienced with mechanical clips. As a result of the foregoing changes, improvements and modifications to CFV modules, increased reliability and longevity of CFV modules could be realized, although occasionally at the expense of additional parts and manufacturing steps.

Accordingly, a capillary force vaporizer according to another aspect of the instant disclosure is a device or method for generating pressurized vapor from unpressurized liquid, comprising:
1. a porous member 120 further comprising a capillary network, a surface for receiving liquid and a vaporization area in which vapor is produced from the liquid;
2. a heater 510 or 710 for conveying heat to the porous member;
3. a heat distributor 610 disposed between the porous member and the ring heater for distributing heat uniformly to the vaporization area of the porous member; and
4. means for situating the ring heater, heat distributor and porous member 120 in heat-exchanging contact with one another and for providing compression among these elements;

wherein the heat distributor includes at least one orifice, the orifice selected from the group comprising a hole, a channel, a plurality of holes, a plurality of channels, and combinations of any of the foregoing.

As an improvement to the CFV module shown at 600 in FIG. 6., a CFV as contemplated herein may also be realized by using either a grooved porous member in combination with a fibrous wick or a grooved elongated non-fibrous wick. Thus, a different capillary force vaporizer according to yet another aspect of the instant disclosure is a device or method for generating pressurized vapor from unpressurized liquid, comprising:
1. a porous member 120 further comprising a capillary network, a surface for receiving liquid and a vaporization area in which vapor is produced from the liquid and further including a plurality of grooves or channels for the collection and pressurization of vapor that is produced from the liquid;
2. a heater 710 for conveying heat to the porous member;
3. a heat distributor 610 disposed between the porous member and the heater for distributing heat uniformly to the vaporization area of the porous member; and
4. means for situating the ring heater, heat distributor and porous member 120 in heat-exchanging contact with one another and for providing compression among these elements.

In yet another aspect, an improved capillary force vaporizer may comprise:
1. a non-fibrous wick 410 comprising a capillary network, a surface for receiving liquid and a vaporization area in which vapor produced from the liquid is collected and pressurized. The wick may have a smooth upper surface, or may further include a plurality of grooves or channels for the collection and pressurization of vapor that is produced from the liquid;
2. a heater 710 for conveying heat to the porous member;
3. a heat distributor 610 disposed between the porous member and the ring heater for distributing heat uniformly to the vaporization area of the porous member; and
4. means for situating the ring heater, heat distributor and porous member 120 in heat-exchanging contact with one another and for providing compression among these elements.

### Improved Temperature Control

An additional feature that may be included with the CFVs described herein is an electrical resistor, thermocouple or thermistor, which can be used for temperature sensing and control of the CFV heater. With reference to FIG. 9, therefore, the partial CFV as shown at 900 includes optional temperature sensor 912 with leads 910. Temperature sensor 912 can be incorporated into optional groove 408, which is cut into a surface or an edge of upper surface 402 of upper portion 412 of grooved elongated non-fibrous wick 410. As will be understood by those knowledgeable in the relevant art, optional groove 408 may also be incorporated in upper portion 302 of non-grooved long ceramic wick 310. The actual placement of a temperature sensor or groove 408 in any of the wicks discussed herein is not critical. For example, a temperature sensor may be placed on any surface of the components of a CFV module, whether or not it is situated in a groove. For example, a temperature sensor may be located on a surface of the heater. The purpose for including a temperature sensor with a CFV is to monitor temperature changes at the heater as closely as possible. The only criteria for location of a temperature sensor, therefore, is to position it on or as close as possible, to a surface of the heater.

Past CFV devices have relied on a microprocessor to control the voltage and measure the current at the heater, in order to determine its resistance. From the heater's resistance, the temperature of the heater can be estimated, although it was found that the variability in measuring heater temperature using the resistive technique is ±10° C. with an aqueous feed. In the course of the instant work, it was found that if a CFV operating with aqueous feed is fitted with a temperature sensor such as a thermistor, the thermistor could be used to monitor the heater temperature and help maintain a consistent temperature while the device was running. That is, a temperature sensor can ensure that the temperature at which the heater is maintained is kept constant and as close as possible to the 100° C. (212° F.) boiling point of water. A circuit can be set to turn off if the thermistor senses temperatures at the heater in excess of 100° C., indicating that the CFV has exhausted its aqueous feed supply. Similarly, a circuit can be set to power the heater if the thermistor senses temperatures at the heater less than 100° C. The use of a temperature sensor with the CFVs described herein has permitted a much more reliable and simple manner for monitoring the temperature of the heater, obviating the need for a microprocessor, as had been used with prior CFV devices. Rather than require feedback relays and a microprocessor for control and communication with heaters having exposed heating traces and soldered electrical leads, the temperature monitoring and control in the CFVs described herein is more straightforward.

### Example 3

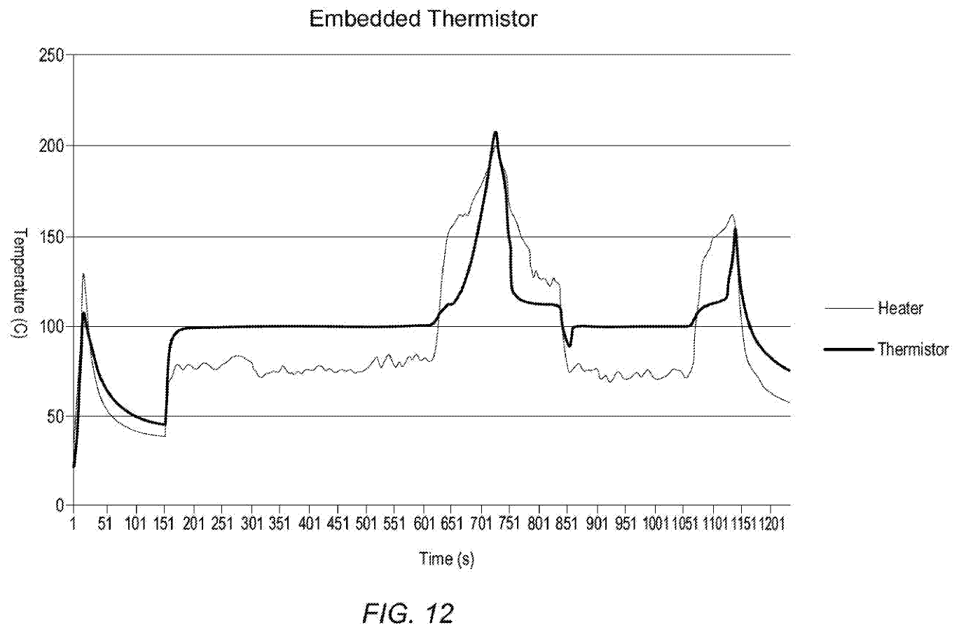

In this study, two different techniques for monitoring temperatures of CFVs are shown and compared in the graph presented in FIG. 12. FIG. 12 shows a graph of temperature (° C.) versus time (sec) for a thermistor embedded in an extended non-fibrous wick (heavy line) compared to a thermocouple affixed directly to the top surface of the heater (light line). The heavy trace shows that the temperature measured by the thermistor is fairly steady and tracks very well with the temperature of the ceramic wick at nearly exactly 100° C. while there is aqueous fed to the CFV. The spikes that begin after 601 seconds and again after 1051 seconds indicate what can happen when the CFV runs out of feed. In the absence of additional liquid to cool the heater, the CFV will experience a temperature excursion and heat up.

Also, from FIG. 12, it can be seen that monitoring the temperature of a heater via a thermocouple in direct contact with a surface of the heater is more sensitive, but results in greater temperature fluctuations, as evidenced by the relative amount of jaggedness of the lighter line in FIG. 12. By comparison, the variability in temperature measurements using a thermistor were found to be ±1° C. Note that the heavy trace in FIG. 12 is almost steady at 100° C. as evidenced by the nearly consistent tracking of the horizontal line during operation. Use of a temperature sensor therefore surprisingly provided an acceptable and more straightforward technique for monitoring heater temperatures during CFV operation. This surprising advantage had not been contemplated with prior CFV devices. Moreover, including a temperature sensor at the heater showed that improved temperature control and comparable responsiveness could be achieved in CFV devices with fewer components and simpler electronics than previously used.

As indicated above, an alternate configuration for a CFV as contemplated herein is shown in FIG. 13 at 1300. CFV 1300 includes heater 710 with orifice 708, and attachment site 1312 on top surface 702 of heater 710 for power leads 706. Adjacent to and in heat-exchanging contact with heater 710 is non-fibrous long wick 410, which again includes upper portion 412 and lower portion 414, portions of which are also shown via phantom lines within holder 210. Holder 210, which encircles wick 410 and provides ledge 212 (not shown) upon which upper portion 412 of wick 410 rests, provides means to engage mechanical force generator 1304 as follows. Holder 210 includes one or more tabs or other form of contact means 1302 disposed towards an exterior portion of holder 210. As illustrated as having one configuration in FIG. 13, tabs 1302 are present in pairs at diametrically opposed locations along an upper circumference of holder 210. Mechanical force generator 1304 includes deflectable portion or engagement means 1308 that contact upper or top surface 702 of heater 710. Lower portion 1306 of engagement means 1308 includes arms 1309 to engage tabs 1302 of holder 210. As depicted in FIG. 13, arm 1309 permits engagement of mechanical force generator 1304 with holder 210 at a portion of tab 1302 that is disposed in a direction opposite to the top surface 702 of heater 710. In this manner, mechanical force generator 1304 provides compressive force as between upper portion 412 of non-fibrous wick 410 and heater 710. Also included in FIG. 13 is temperature sensor 912 with leads 910. Temperature sensor 912 is compressively held onto upper surface 702 of heater 710 via deflectable span 1310, which, in this instance, joins two engagement means 1308 at opposite ends of mechanical force generator 1304.

One advantage of using a deflectable mechanical force generator such as 1304 as shown in FIG. 13 to position temperature sensor 912 onto top surface 702 of heater 710 and therefore the CFV is that it is not necessary to use epoxy, gluing or fixing means to attach the temperature sensor to the CFV. Simple mechanical force has been shown to provide sufficient and appropriate attachment function. Furthermore, positioning temperature sensor 912 on top of heater 7170 using mechanical force generator 1304 obviates the need for modification of wick 410 as per additional milling, drilling or cutting to fashion thermistor groove 408 (not shown in FIG. 13; but see FIG. 9), as part of wick 410. This, in turn, simplifies the production of CFV components, requires fewer steps and can result in a corresponding decrease in materials, manufacturing and assembly costs. Further, as will be readily appreciated by those knowledgeable in the relevant field(s), the sample “hook-bar-hook” type of mechanical force generator depicted at 1304 in FIG. 13 may feature any of a wide number of variations in shape, configuration, number of tabs, arms, attachment means, etc. Further modifications and configurations of mechanical force generator components may therefore be contemplated for use with the CFV modules described herein and are therefore regarded as part of the scope of the invention contemplated by the instant disclosure.

In still a further aspect, therefore, a capillary force vaporizer according to another configuration may be regarded as a device or method for generating pressurized vapor from unpressurized liquid, comprising:
1. a grooved non-fibrous wick 410 comprising a capillary network, a surface for receiving liquid and a vaporization area in which vapor is produced from the liquid and further including a plurality of channels for the collection and pressurization of vapor that is produced from the liquid;
2. a heater 710 including an embedded heating element for conveying heat to the grooved wick, the heater further including at least one orifice for release of the vapor at a velocity greater than zero;
3. means for situating the heater and grooved wick in heat-exchanging contact with one another and for providing compression among the heater and wick; and
4. optionally, means for monitoring and controlling the temperature of the heater using a temperature sensor. The temperature sensor may be situated at an upper surface of the grooved wick in heat-sensing contact with the embedded heater. Different locations for the temperature sensor are also possible.

A method for the vaporization of liquids as contemplated herein may therefore be regarded as comprising: A method for the vaporization of a liquid to a pressure greater than that of a liquid feed as contemplated herein may therefore be regarded as comprising:
1. providing a liquid feed to a portable thin film vaporization device, the liquid characterized as having a first pressure; and
2. vaporizing thin films of the liquid to produce a vapor, the vapor characterized as having a second pressure; wherein the second pressure is greater than the first pressure; and wherein the portable vaporization device comprises:
   a) a long, non-fibrous wick comprising a capillary network, a surface for receiving liquid and a vaporization area in which vapor produced from the liquid is collected and pressurized;
   b) an optional heat distributor;
   c) a heater for conveying heat to the wick, the heater also including at least one orifice for the release of the pressurized vapor; and
   d) a mechanical force generator for providing compression among the heater, optional heat distributor and non-fibrous wick.

The present invention has been described above in detail with reference to specific embodiments, Figures, Graphs and examples. These specific embodiments should not be construed as narrowing the scope of the disclosure, but rather as illustrative examples. It is to be further understood that various modifications and substitutions are anticipated and may be made to the described vaporization modules and devices, as well as to materials, methods of manufacture and use, without departing from the broad spirit or scope of the invention contemplated herein. The invention is further illustrated and described in the accompanying figures and the claims, which follow.

What is claimed:

1. A portable thin film vaporizer device for the generation of pressurized vapor from unpressurized liquid, comprising:
a) a non-fibrous wick comprising a capillary network, a surface for receiving liquid and a vaporization area in which vapor produced from the liquid is collected and pressurized; and
b) a heater for conveying heat to the non-fibrous wick, the heater also including at least one orifice for the release of the pressurized vapor;
wherein the heater is situated in heat-exchanging contact with the non-fibrous wick.

2. The vaporizer of claim 1, wherein the non-fibrous wick includes a plurality of grooves for the collection and pressurization of vapor that is produced from the liquid, and further wherein the heater includes an embedded heating element.

3. The vaporizer of claim 2, further comprising:
c) at least one of:
1) means for situating the non-fibrous wick in heat-exchanging contact with the heater;
2) means for providing compression among the heater and wick;
3) both 1) and 2); and
d) a heat distributor.

4. The vaporizer of claim 3, wherein the heat distributor further comprises a plate.

5. The vaporizer of claim 4, wherein the heat distributor is comprised of material selected from the group comprising stainless steel, metal, heat-conducting ceramics, heat-conducting polymers, and combinations of any of the foregoing.

6. The vaporizer of claim 5, wherein the distributor is comprised of stainless steel.

7. The vaporizer of claim 3, further comprising:
e) a temperature sensor.

8. The vaporizer of claim 3 that is capable of vaporizing at least 2.0 g/min water.

9. A portable thin film vaporizer device for the generation of pressurized vapor from unpressurized liquid, comprising:
a) a non-fibrous wick comprising a capillary network, a surface for receiving liquid and a vaporization area in which vapor produced from the liquid is collected and pressurized;
b) a heat distributor; and
c) a heater for conveying heat to the wick, the heater also including at least one orifice for the release of the pressurized vapor;
wherein the heat distributor is situated in heat-exchanging contact with the heater and the wick.

10. The vaporizer of claim 9, wherein the heat distributor comprises a plate.

11. The vaporizer of claim 10, wherein the heat distributor is comprised of material selected from the group comprising stainless steel, metal, heat-conducting ceramics, heat-conducting polymers, and combinations of any of the foregoing.

12. The vaporizer of claim 9, further comprising:
d) at least one of:
1) means for situating the heat distributor in heat-exchanging contact with the heater and the wick;
2) means for providing compression among the heater, heater distributor and non-fibrous wick; or
3) both 1) and 2).

13. The vaporizer of claim 9, wherein the heat distributor includes no orifice, one orifice or a plurality of orifices, further wherein the orifice may comprise a hole, groove, slot, channel, a plurality of any of the foregoing or a mixture of any of the foregoing, for the collection and pressurization of vapor that is produced from the liquid, and further wherein the heater includes an embedded heating element.

14. The vaporizer of claim 13, further comprising:
e) a temperature sensor.

15. A method for vaporizing a liquid to a pressure greater than that of a liquid feed, comprising:
a) providing a liquid feed to a portable thin film vaporization device, the liquid characterized as having a first pressure; and
b) vaporizing thin films of the liquid to produce a vapor, the vapor characterized as having a second pressure;
wherein the second pressure is greater than the first pressure; and
wherein the portable vaporization device comprises:
1) a non-fibrous wick comprising a capillary network, a surface for receiving liquid and a vaporization area in which vapor produced from the liquid is collected and pressurized;
2) an optional heat distributor;
3) a heater for conveying heat to the wick, the heater also including at least one orifice for the release of the pressurized vapor; and
4) a mechanical force generator for providing compression among the heater, optional heat distributor and wick.
